(19) United States
(12) Patent Application Publication
(10) Pub. No.: US 2004/0087974 A1
(43) Pub. Date: May 6, 2004

(54) Suture welding device and method of welding

(76) Inventor: Edward S. Bittar, Indialantic, FL (US)

Correspondence Address: ROGER M. RATHBUN, 13 MARGARITA COURT, HILTON HEAD ISLAND, SC, 29926, US

(21) Appl. No.: 10/244,348

(22) Filed: Sep. 16, 2002

(51) Int. Cl. A61B 17/10

(52) U.S. Cl. 606/139

(57) ABSTRACT

A device and method to carry out the welding of suture strands joining tissue of a patient. The device has an elongated housing with a distal opening and a proximal opening for the suture strands to pass through the device generally along the elongated axis of the device. A pair of welding plates surround the suture strands passing through the device and can be activated to provide heat to weld the strands together. One of the welding plates is movable by means of an externally accessible button for the physician to move that welding plate between an open position where the strands can easily be threaded between the welding plates and a closed position where the suture strands are held tightly together between the welding plates and the weld can be effected.

### BACKGROUND OF THE INVENTION

The present invention relates to a device and a method of securing strands of a medical suture together, and, more particularly, to a device and method of welding together individual suture strands.

There are, of course, many surgical procedures that require suturing of the tissue of the patient to close an opening in the patient and/or to reattach tissue to bone or other soft tissue of the patient. In the majority of cases, the strands of the suture are tied by the physician to secure the suture in place such that the suture strands are secured together in close proximity to the tissue and, preferably, held together against the particular tissue while the healing process is taking place. In most cases, multiple knots are required to prevent unraveling and to provide a secure attachment of the suture strands.

The typical monofilament suture is stiff and, in some surgical settings, the material can be difficult to work with, especially when tying knots. Further, there are simply times that the tying of a knot in a suture to secure the same can be inconvenient due to the need to make the knot in a relatively inaccessible location.

In the main, the material of a suture strand is a monofilament polymer plastic that is susceptible to welding as a means of securing the ends of a suture together and such welding devices have been proposed and are currently in use by physicians. For example, there is a commercial welding device that can be used to secure the strands of a suture together marketed by the Axya Company that uses ultrasonic energy to carry out the welding procedure by means of a hand held instrument. An example, by that same company, of a fused loop of a suture is shown and described in U.S. Pat. No. 5,893,880 of Egan et al.

Other welding devices that have been disclosed to carry out the welding of sutures are shown and described in U.S. Pat. No. 4,662,068 of Polonsky and U.S. Pat. No. 6,077,277 of Mollenauer et aL In the case of the latter patents, the devices feature some clamping means and/or are cumbersome devices that may not be advantageous if the weld is desired within a relatively inaccessible area of the body such as would be likely with the use of endoscopic or arthroscopic procedures.

One of the difficulties with the welding of suture strands is that it is necessary to locate the weld snugly or tightly secured against the tissue so that the weld can secure the suture in a manner that the sides of the tissue are brought together in a close, abutting relationship. As such, it is important that the actual strands of the suture be pulled in a manner that the tissue on both sides of a tear, for example, are pulled together and not spread apart as the weld is made.

In addition, there is a further problem in the making of welds in suture strands in that the strands or loose ends of the suture are normally positioned in close, abutting or touching relationship parallel to each other in making the weld and the application of energy in that area causes the melting of the monofilament suture strands to bond those strands together. The consequent weld, therefore, is accomplished along the length of the suture strands and the completed weld is subject to a tearing force that can separate those strands after the completion of the weld itself. As such, there is a weakness of the completed weld if the suture strands are subjected to a tearing force since the direction of a tearing force can pull the strands apart progressively along the length of the weld.

Accordingly, it would be advantageous to have a device that can facilitate the aforedescribed welding of medical sutures where the device can be used to weld suture strands such that the weld itself is located in a close relation to the tissue and the use of the device encourages the tissue to be joined tightly together. It would be further advantageous to provide a suture welding device that produces a strong bond between the individual strands of the suture. It would be still further advantageous to provide a suture welding device that is dimensioned sufficiently small so as to be utilized in relatively inaccessible areas of the patient.

### SUMMARY OF THE INVENTION

Accordingly, the present invention relates to a suture welding device and a method to carry out the welding of suture strands that provide a strong, secure affixing together of those suture strands and which can also be utilized to secure the suture in relatively inaccessible areas of the patient easily and conveniently for the physician.

With the present invention, there is a suture welder device to affix monofilament suture strands together and which comprises an elongated housing that is conveniently sized to be a hand held device and can be used by, and indeed is well suited for, the physician even in remote locations within the patient such as the joining of tissues in the abdomen or pelvis of a patient, or in the repair of a rotator cuff of a patient's shoulder or the meniscus of a patient's knee.

Thus the elongated housing of the subject suture welding device has a distal end and a proximal end. The housing can preferably be constructed of a metal material and intended for reuse by the physician or may be constructed of plastic to be a disposable device. The elongated housing can be cylindrical and is dimensioned sufficiently small to be positioned within relatively inaccessible areas of the patient. Located in the elongated housing is a button attached to an actuator arm that closes the gap between welding plates and a switch to activate the power to the working elements and the power may be supplied by some external source of energy or, alternatively, the housing may contain batteries and be self-powered.

The distal end is a blunt, rounded tip to allow the tip to allow easy entry through the skin, other tissue planes, and body cavities relatively atraumatically, and allow the tip be effective at most angles of approach. There is, at the distal end, a distal opening that is preferably formed coaxial with the main longitudinal axis of the elongated housing and the distal opening communicates with a passageway that is formed in the housing leading to a further opening, located proximally inward a distance displaced away from the distal end and which can be conveniently located in the side wall of the device. By means of the distal and proximal openings with the passageway between the openings, the physician can introduce or thread the suture strands to be welded into the distal opening, through the passageway and out of the proximal opening.

Thus, as a feature of the invention, the orientation of the free ends of the suture strands can be pulled directly outwardly from the tear in the tissue that is being repaired and the suture strands pass through the device generally along the longitudinal axis of the welding device so that the pulling of the free ends of the suture strands encourages the closing of the tear in the tissue. The ultimate weld joining the strands of suture can therefore be located tightly against the tissue with the tear naturally pulled closed.

In addition, the longitudinally directed pulling force outwardly from the tear in the tissue creates less lateral stress on the suture strands themselves than if they were pulled in the lateral direction. Further, with the longitudinal orientation of the passageway, it is possible to twist together the strands of suture prior to welding those strands together to proportionally increase the amount of surface of the monofilament suture strands in contact with each other for any measured length.

There are also a pair of welding plates located at the distal end of the suture welder housing and the welding plates are located at the periphery of the passageway such that any suture strands that pass through the passageway must also pass between the welding plates. Surrounding the welding plates is an insulating material such that the insulated distal end can come in contact with the tissues during the welding process without causing damage to the tissues.

Normally, the welding plates are spaced apart sufficiently to allow the suture strands to pass through the passageway, however, the physician can cause at least one of the welding plates to move with respect to the housing to force the suture strands against each other in a tight abutting relationship.

In the preferred embodiment, the relative movement between the welding plates is accomplished by constructing one of the welding plates to be immobile within the housing while the other welding plate is movable with respect to the housing by means of a button that is accessible to the physician along the exterior of the housing.

Accordingly, when the physician desires to weld the suture strands passing through the passageway in the housing, the external button is depressed so as to force the welding plates against the suture strands and the sutures against each other. Energy is then applied to melt the suture strands to create the weld between those strands. The energy employed to melt the suture strands can be a variety of energies including, but not limited to, resistance heating, RF energy or ultrasonic energy. Upon completion of the weld, the free ends of the remaining suture strands can be trimmed to create a weld of the suture that is tightly maintained against the tissue.

In a further embodiment, there may be a plug or sleeve comprised of a polymeric material that is located in the passageway and in close proximity to the welding plates. The plug or sleeve has a passage therethrough and the suture strands pass through that passage as the suture strands are threaded through the passageway in the suture welding device housing.

As such, when the energy is applied to the welding plates, the consequent heat applied to the suture strands also causes melting of the polymeric plug such that the ultimate weld welds the suture strands together but additionally those suture strands are surrounded by the plug that is also melted to those suture strands. That resultant weld is, therefore, much stronger by the addition of the plug material and also serves to alleviate the problem with normal welds that can be peeled apart along the length of the longitudinal weld to destroy a finished weld.

In addition, with the addition of a plug or sleeve to the weld, the lengths of the suture strands that are actually incorporated into the weld can be shortened without reducing the overall strength of the weld. Thus, with the plug or sleeve embodiment, the weld plates can be reduced in size along with the size of the housing itself and which can make the suture welding device even smaller to carry out the welding process in the relatively inaccessible areas of the patient.

These and other features and advantages of the present invention will become more readily apparent during the following detailed description taken in conjunction with the drawings herein.

### BRIEF DESCRIPTION OF THE DRAWINGS

FIG. 1 is a side, cross sectional view of the suture welding device of the present invention,

FIG. 2 is a lateral cross sectional view of the device taken along the lines 22 of FIG. 1;

FIG. 3 is an enlarged side, cross sectional view of the distal end of an alternate embodiment of the welding device;

FIG. 4 is an enlarged side cross sectional view of the distal end of a further embodiment of the present invention;

FIGS. 5A-5C are a bottom view, a side view and a cross sectional view, respectively, of a plug used with the FIG. 4 embodiment;

FIG. 6 is a schematic view of the present suture welding device used in the repair of a meniscus of a patient's knee;

FIG. 7 is a schematic view of the present suture welding device used in the repair of a rotator cuff of a patient's shoulder;

FIG. 8 is a schematic view of a further step in the repair procedure of FIG. 7;

FIG. 9 is a schematic view of the completed weld of the procedure of FIGS. 7 and 8; and

FIG. 10 is a schematic view of the trimming of free ends of suture strands after completion of the welding procedure using the present invention.

### DETAILED DESCRIPTION OF THE INVENTION

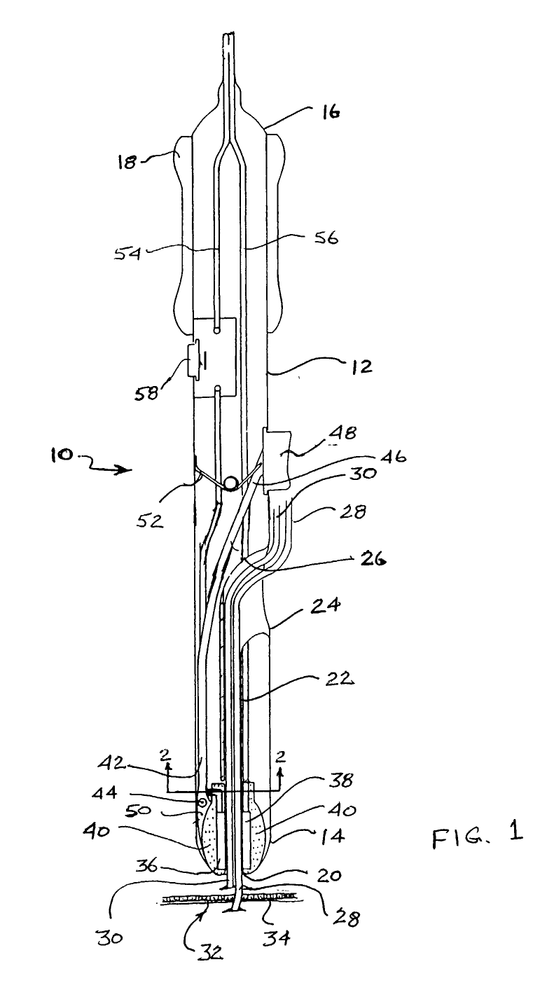

Referring now to FIG. 1, there is shown a side cross sectional view of a suture welding device 10 constructed in accordance with the present invention. As can be seen, the device 10 comprises an elongated housing 12 having a longitudinal axis and which is preferably circular in cross section. The elongated housing 12 has a distal end 14 and a proximal end 16. In general, the distal end 14 is of a blunt, rounded shape to allow the device 10 to be more easily and more atraumatically introduced through tissue planes into a body cavity and to be effective at most angles of approach. A handle 18 is provided at the proximal end 16 of the welding device 10 for the convenience of the physician in utilizing the device 10 to weld a suture.

A distal opening 20 is formed in the housing 12 and a passageway 22 is formed in the housing 12 beginning at that distal opening 20 and extending generally along the longitudinal axis of the housing 12 to a proximal opening 24 that is formed in the side wall of the housing 12 and is displaced proximally away from the distal end 14 of the housing. While the exact distance that the proximal opening 24 is located displaced away from the distal end 14 may vary, it is advantageous that the passageway 22 at least generally follow the longitudinal axis of the elongated housing 12 from the distal opening 20 proximally inwardly as will later be explained.

While the proximal opening 24 is preferred to be formed in the side wall of the housing 12, it is possible for the proximal opening 24 to be at the very proximal end 16 of the housing 12. In the preferred embodiment, as shown in FIG. 1, the distal opening 20 is coaxial with the longitudinal axis of the housing 12 and there is an arcuate transition surface 26 between the longitudinal portion of the passageway 22 and the proximal opening 24.

Thus, in the use of the suture welding device 10, the suture strands 28, 30 form a loop through the tissue 32 around the tear 34 to be repaired in the normal course of the suturing procedure by a physician and those suture strands 28, 30 are threaded through the distal opening 20, then through the passageway 22 to emerge from the proximal opening 24 where the suture strands 28, 30 can be grasped by the physician and tension exerted in the carrying out of the welding procedure as will become clear. The threading process is simplified by the transition surface 26 that is curved so that the suture strands 28, 30 can easily be push around the transition surface 26 to emerge from the proximal opening 24.

With the use of a distal opening 20 that is coaxial with the longitudinal axis of the elongated housing 12, it can be seen that the suture strands 28, 30 can be brought out together and directed straight into the passageway 22 so that the tear 34 is naturally brought together rather than forced apart. In addition, with the particular orientation of the passageway 22, the suture strands 28, 30 can be twisted by the physician to improve the ultimate weld.

Located at or proximate the periphery of the passageway 22 at the distal end 14 of the housing 12, there are a pair of welding plates and, in the preferred embodiment, those welding plates consist of a movable welding plate 36 and a stationary welding plate 38, the latter being fixed in its location with respect to the housing 12. The former, the movable welding plate 36, is movable with respect to the stationary welding plate 38 so that the distance between the welding plates 36, 38 can be changed between an open position where the welding plates 36, 38 are spaced relatively far apart to allow the suture strands 28, 30 to be threaded into and through the passageway 22 as well as between the welding plates 36, 38 and a closed position wherein the welding plates 36, 38 are moved into a close together position wherein suture strands 28, 30 are pressed tightly together by the welding plates 36, 38.

There is an insulating material 40 external of the welding plates 36, 38 so that the heat from the welding plates 36, 38 is confined to be directed inwardly toward the suture strands 28, 30 and not to a great extent travel outwardly through the housing 12.

Accordingly, there is a means to move the movable welding plate 36 to cause the welding plates 36, 38 to be moved between the open and the closed positions, and, as shown, the means includes an actuator arm 42 that can be pivotally affixed to the housing 12 by a pivot 44. At the proximal end 46 of the actuator arm 42, there is a button 48 that is accessible at the exterior of the housing 12. The button 48 can be depressed as desired by the physician to rotate the actuator arm 42 about the pivot 44 to move the distal end 50 of the actuator arm 42 towards the movable welding plate 36 to move the movable welding plate 36 toward the stationary welding plate 38.

As such, by depressing the button 48, the physician can move the movable welding plate 36 so that the welding plates 36, 38 can be moved between the open and closed positions. A bias is provided by a spring 52 to bias the button 48 outwardly or to bias the movable welding plate 36 toward the open position of the welding plates 36, 38.

The energy to heat the welding plates 36, 38 is supplied by means of electrical wires 54, 56 that both exit the suture welding device 10 at the proximal end 16 and there is an on/off switch 58 that can be depressed by the physician to make a momentary contact to transmit the energy to the welding plates 36, 38. Thus, the physician can depress the button 48 to bring the welding plates 36, 38 to their closed position sandwiching the suture strands 28, 30 therebetween and then depress the on/off switch 58 to cause the energy to reach the welding plates 36, 38 to heat the welding plates 36, 38 and thereby cause the weld to be effected through the melting of the suture strands 28, 30. The energy can be any one of various conventional energy sources including electrically powered resistance heating, RF energy or ultrasonic energy to heat the welding plates 36, 38.

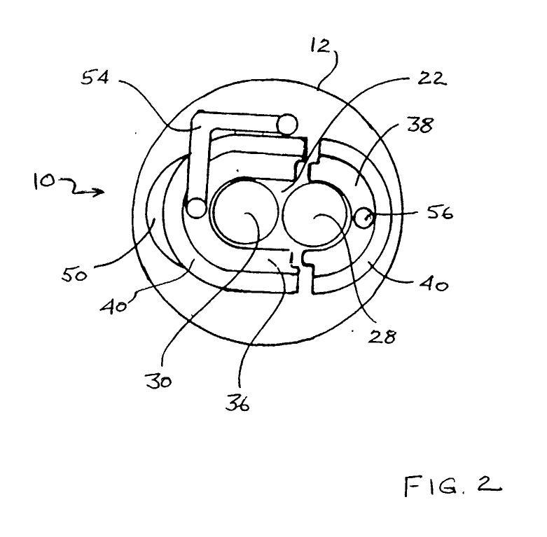

Turning now to FIG. 2, there is shown a cross sectional view of the suture welding device 10 and illustrating the housing 12 with the passageway 22 within that housing 12. The welding plates 36, 38 are curved so as to capture the suture strands 28, 30 therebetween to align the suture strands 28, 30 generally parallel to each other. In FIG. 2, the welding plates 36, 38 are in the open position where the suture strands 28, 30 can be threaded through the passageway 22, however, as can be seen, when the proximal end 50 of the actuator arm (not shown in FIG. 2) moves the movable welding plate 36, those suture strands 28, 30 can be brought into a tight abutting relationship to carry out the welding of the suture strands 28, 30.

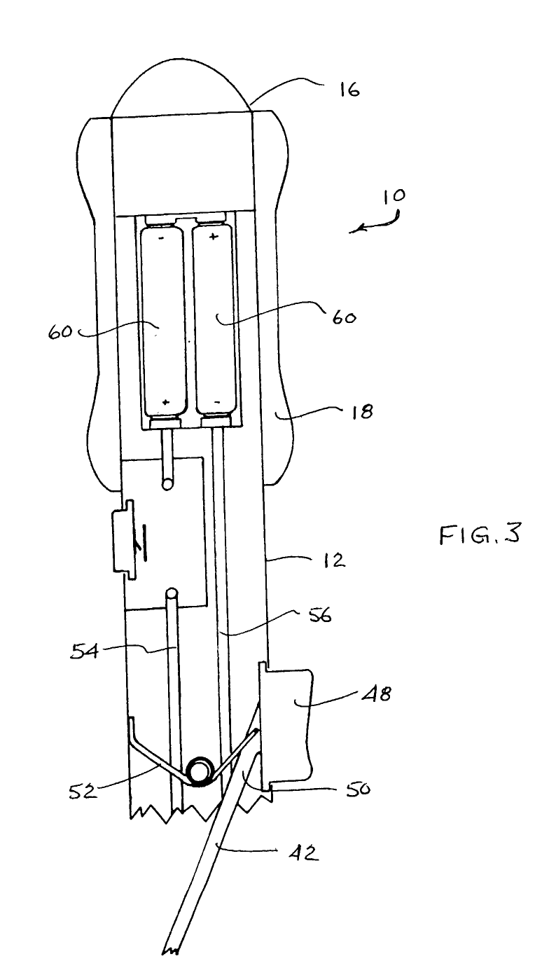

In FIG. 3 there is an enlarged side cross sectional view of the proximal end 16 of the suture welding device 10. In this embodiment, there is an internal source of energy shown as batteries 60 to power the device 10 such that the embodiment is self-contained and the entire device 10 can be held and manipulated by the physician in carrying out the welding procedure.

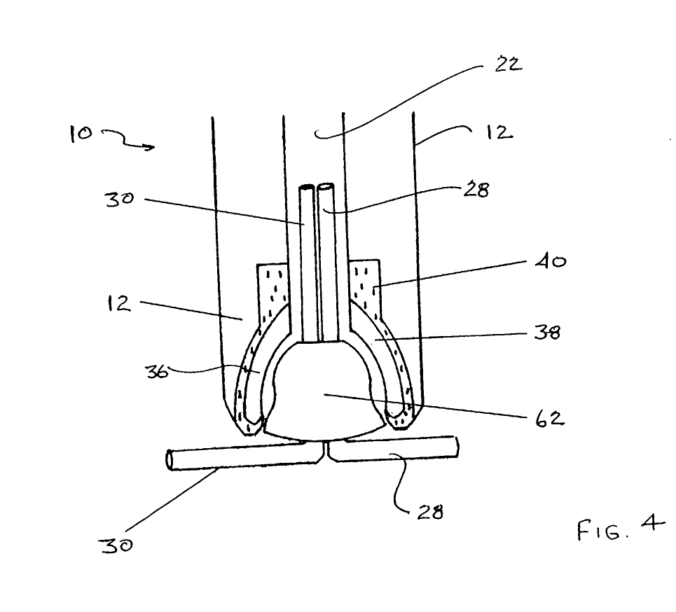

Turning now to FIG. 4, there is shown a side cross sectional view of an alternative embodiment where there is a bell-shaped plug 62 that is located within the passageway 22 and, as can be seen, the suture strands 28, 30 pass through the plug 62 in close proximity to where the suture strands 28, 30 pass by the welding plates 36, 38. Accordingly, when the weld is produced by energizing the welding plates 36, 38, the plug 62 is also heated along with the suture strands 28, 30.

The plug 62 is made of a polymeric material that is readily meltable at the temperature produced by the welding plates 36, 38 so that the plug 62 is melted along with the suture strands 28, 30. In the preferred embodiment, the plug 62 is made of the same monofilament material that is used for the suture strands 28, 30 so that the plug 62 melts at the same temperature as the suture strands 28, 30 when the weld is completed.

As an alternative, instead of a bell shaped plug 62, a ring shaped plug, or a cylindrical or other shaped sleeve of a polymeric material could also be used to strengthen the ultimate weld in the suture strands 28, 30. In either case, the use of the plug 62, or sleeve, serves to strengthen the weld and the plug 62 or sleeve is actually incorporated into the weld replicating the characteristics of a spot-weld. The additional mass of material prevents the peeling effect, that is, where the welded suture strands can otherwise be peeled apart at the apex of the weld and the peeling continue until the welded strands are completely parted.

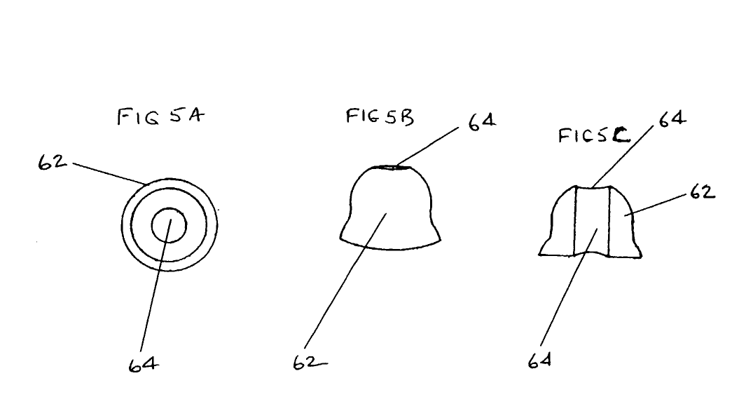

In FIGS. 5A, 5B and 5C, there is shown a bottom view, a side view and a cross sectional view, respectively, of the bell shaped plug 62 used with the invention and, as can be seen, the bell-shaped plug 62 has a central passage 64 that passes through the plug 62 so that the suture strands 28, 30 (FIG. 1) can readily pass through the plug 62 as the suture strands 28, 30 are threaded through the passageway 22.

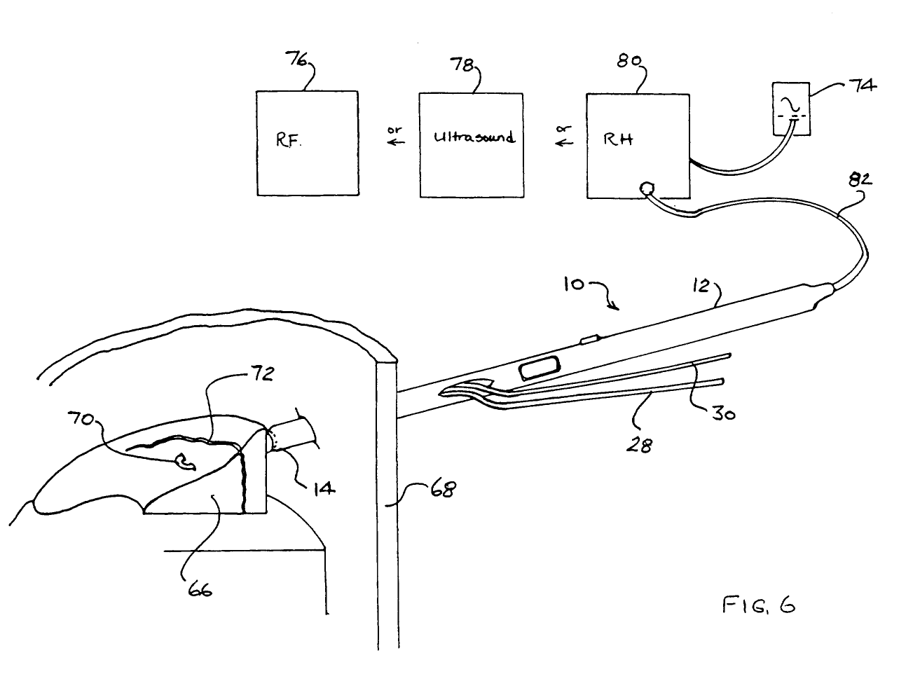

Turning now to FIG. 6, there is a schematic view of a suture welding device 10 being used to carry out the welding of suture strands 28, 30 in the repair of the meniscus 66 of a patient's knee. In FIG. 6 the suture welding device 10 passes through the skin 68 of the patient and the suture strands 28, 30 have been passed through the passageway (not shown) in the housing 12 and have emerged through the proximal opening 24 so that the free ends of the suture strands 28, 30 can be grasped by the physician and tension exerted on those strands as the suture welding device 10 is advanced toward the meniscus 66.

Accordingly, the loop 70 in the suture strands 28, 30 passes through the tear 72 in the meniscus and by drawing the suture strands 28, 30 in a generally longitudinal direction through the housing 12 the suture strands 28, 30 are tensioned in a direction straight outwardly from the outer surface of the meniscus and which serves to pull the tear 72 together rather than apart. As such, the distal end 14 of the suture welding device 10 can be located right at the surface of the meniscus 66 where the weld is carried out so that the suture is held tightly against the meniscus 66 to hold the tear 72 together.

As also shown in FIG. 6, it can be seen that there is an external power source 74 that supplies the power to an energy converter that converts the normal electrical energy to the desired form of energy to the suture welding device 10. For example, the energy converter may convert the energy to RF energy at 76, ultrasonic energy at 78 or radiant energy 80 and that energy, whichever one is used, is carried by the electrical cable 82 to heat the welding electrodes (FIG. 1) to carry out the welding process.

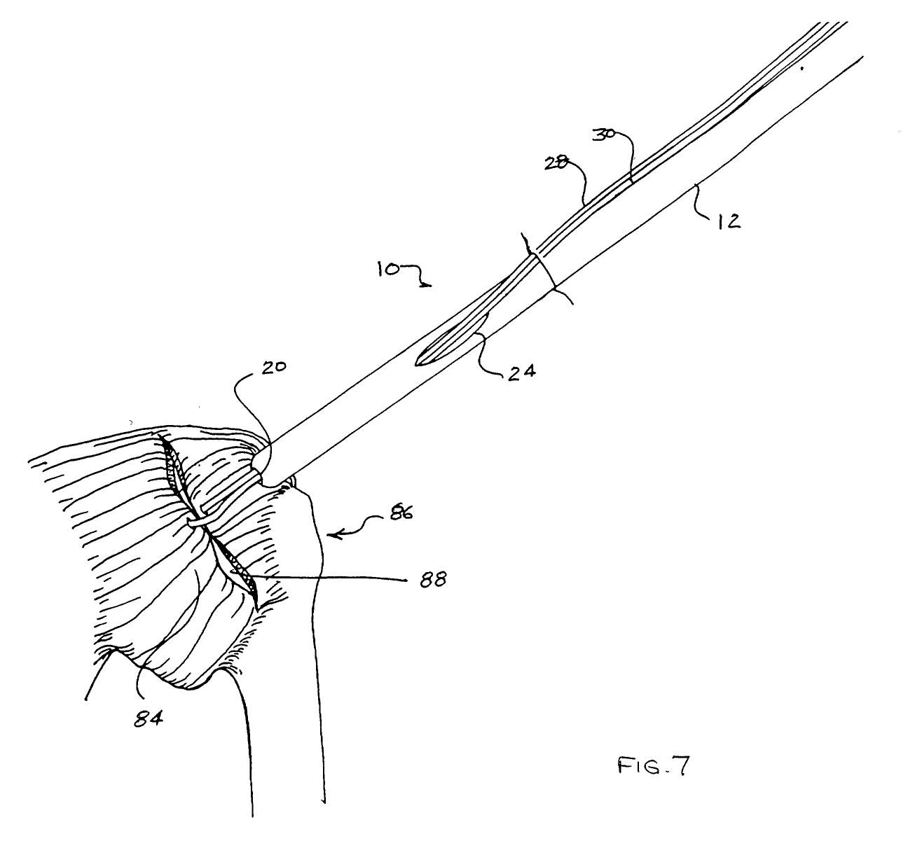

Turning to FIG. 7, there is shown the suture welding device 10 of the present invention being used in the repair of a rotator cuff 84 within the shoulder 86 of a patient. Again, as can be seen, the suture strands 28, 30 enter directly into the distal opening 20 in the suture welding device 10 and pass along the longitudinal axis of the suture welding device 10 to emerge through the proximal opening 24 where the suture strands 28, 30 can be grasped by the physician in manipulating the suture welding device 10 to a position where it is tightly abutting the tear 88 in the rotator cuff 84.

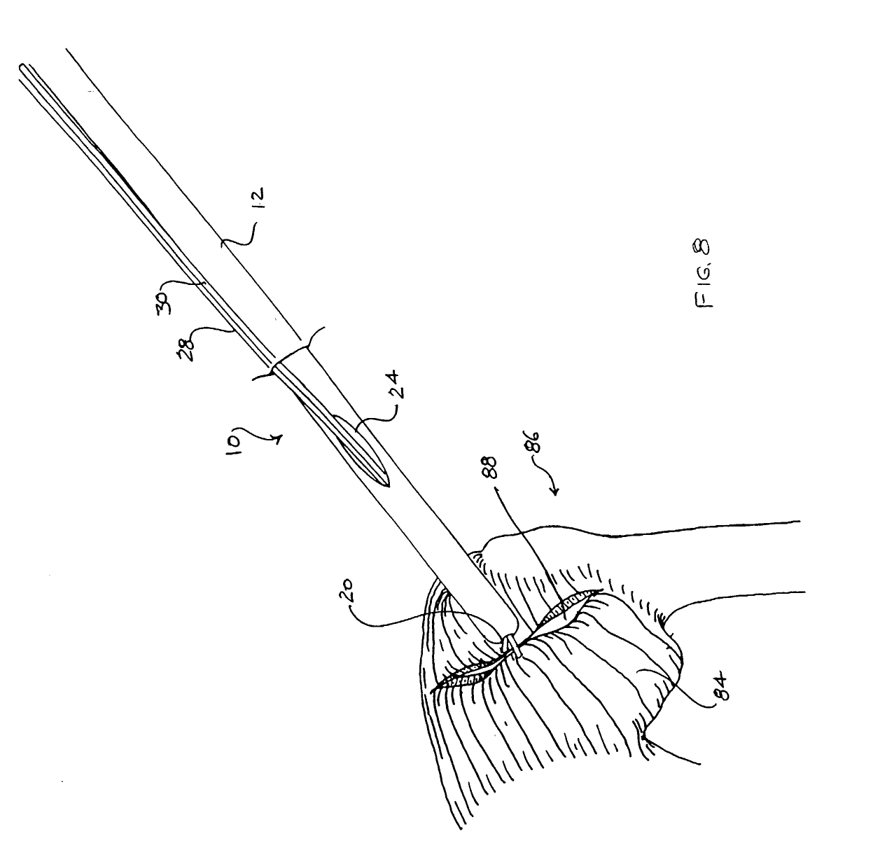

In FIG. 8, there is a further step in the procedure of FIG. 7 and where the suture welding device 10 is close to the rotator cuff 84 due to the direct entrance of the suture strands 28, 30 into the distal opening 20 that is coaxially aligned along the longitudinal axis of the suture welding device 10 so that the suture strands 28, 30 can be pulled directly away from the tear 88 and enter the suture welding device 10.

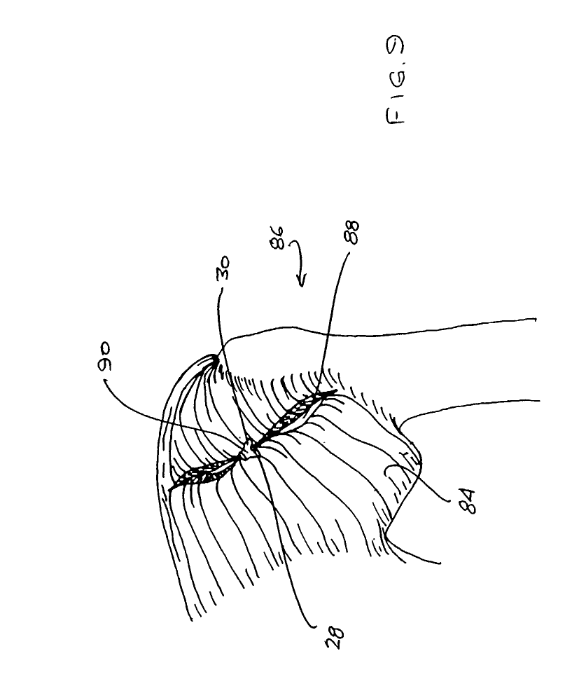

In FIG. 9, there is shown the last step in the procedure of FIG. 7, where the suture strands 28, 30 have been welded together and the free ends trimmed off so that the final weld 90 is closely abutting the tear 88 to hold that tear together to aid in the healing process.

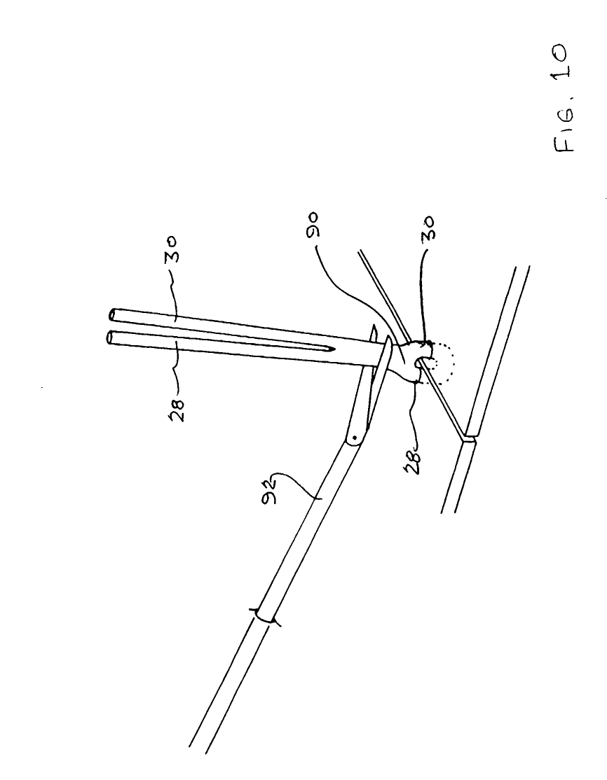

Finally, in FIG. 10 there is a schematic view illustrating the trimming step in the welding procedure and where the suture strands 28, 30 have been welded together and a cutting instrument 92 is used to trim off the excess length of those suture strands 28, 30 to leave the weld in the state as exemplified in FIG. 9.

Those skilled in the art will readily recognize numerous adaptations and modifications which can be made to the suture welding device and method of using the same of the present invention which will result in an improved process and device, yet all of which will fall within the scope and spirit of the present invention as defined in the following claims. Accordingly, the invention is to be limited only by the following claims and their equivalents.

I claim:

1. A device for welding monofilament suture strands together, said device comprising an elongated housing, said housing having a distal end and a proximal end and having a distal opening in said distal end and a proximal opening located proximal to said distal end, said housing having a passageway extending between said distal and said proximal openings to pass suture strands from said distal opening to said proximal opening through said passageway, welding plates located in said distal end of said housing spaced apart to allow suture strands within said passageway to pass between said welding plates, means to move at least one of said welding plates between an open position wherein the suture strands are loosely positioned between said welding plates and a closed position wherein the suture strands are abutted against each other, and means to selectively provide power to said welding plates to heat said welding plates to weld the suture strands together.

2. A device as defined in claim 1 wherein said passageway is substantially oriented along the longitudinal axis of the elongated housing.

3. A device as defined in claim 1 wherein said proximal opening is formed in the side wall of said housing.

4. A device as defined in claim 1 wherein said distal opening is coaxial with the longitudinal axis of the elongated housing.

5. A device as defined in claim 1 wherein said means to move one of said welding plates comprises a button pivotally mounted to the housing and adapted to be activated by a user to move said welding plates between said open and closed positions.

6. A device as defined in claim 1 wherein said button is biased toward the open position of said welding plates.

7. A device as defined in claim 6 wherein said means to move one of said welding plates comprises an actuator arm pivotally affixed to said housing and connected between said button and one of said heating plates, said actuator arm being movable by said button to move one of said welding plates to move said welding plates between said open and closed positions.

8. A device as defined in claim 1 wherein said welding plates are arcuate plates adapted to at least partially surround the suture strands passing between said welding plates.

9. A device as defined in claim 8 wherein said welding plates substantially surround and enclose the suture strands.

10. A device as defined in claim 1 wherein said power for said welding plates comprises power to create resistance heating of said welding plates.

11. A device as defined in claim 1 wherein said power for said welding plates comprises power at RF frequencies.

12. A device as defined in claim 1 wherein said power for said welding plates comprises power at ultrasonic frequencies.

13. A device for welding monofilament suture strands together, said device comprising an elongated housing, said housing having a distal end and a proximal end and having a distal opening in said distal end and a proximal opening located proximal to said distal end, said housing having a passageway extending between said distal and said proximal openings to pass suture strands from said distal opening to said proximal opening through said passageway, welding plates located in said distal end of said housing, a plastic plug located within said passageway and in close proximity to said welding plates, said plug having a passage therethrough, and means to selectively provide power to said welding plates to heat said welding plates to weld said plug and said suture strands together.

14. A device as defined in claim 13 wherein said plug is bell shaped.

15. A device as defined in claim 13 wherein said plug is comprised of the same material as the suture strands to be welded together.

16. A device as defined in claim 13 wherein said welding plates comprise a welding plate that is stationary with respect to said housing and a welding plate that is movable with respect to said housing.

17. A method of welding monofilament suture strands together, said method comprising the steps of:
providing a hand held elongated housing having a distal end and a proximal end with a passageway extending from the distal end to a point displaced proximally away from the distal end, a pair of welding plates located in the distal end of the housing in close proximity to the passageway,
introducing suture strands into the passageway and between the welding plates,
causing the welding plates to contact the suture strands,
heating the suture strands by means of energy from the welding plates to cause melting of the suture strands to weld the suture strands together.

18. A method as described in claim 17 wherein said step of causing the welding plates to contact the suture strands comprises moving at least one of the welding plates.

19. A method as described in claim 17 wherein said step of providing a hand held elongated body with a passageway comprises providing a hand held elongated housing having a passageway that is formed along the longitudinal axis of the elongated housing.

20. A method as described in claim 17 wherein said step of providing a housing having a passageway comprises providing a housing having a passageway containing a plug or sleeve made of a meltable plastic material with a passage therethrough, and said step of introducing suture strands into the passageway comprises introducing suture strands through the passage in the plug.

21. A method as described in claim 20 wherein said step of heating the suture strands comprises heating the plug to cause the plug to melt about the suture strands.

22. A method as described in claim 20 wherein said step of providing a plug or sleeve comprises providing a plug or sleeve made of the same material as the suture strands.
